(19) United States
(12) Patent Application Publication
(10) Pub. No.: US 2008/0175761 A1
(43) Pub. Date: Jul. 24, 2008

(54) Air Sanitizing and Charging/Recharging Base and Rechargeable Device Arrangement

(75) Inventors: Chuck Thur, Broadview Heights, OH (US); Dave Brickner, Concord, OH (US)

Correspondence Address: CALFEE HALTER & GRISWOLD, LLP, 800 SUPERIOR AVENUE, SUITE 1400, CLEVELAND, OH, 44114, US

(73) Assignee: GUARDIAN TECHNOLOGIES LLC, Bedford Heights, OH (US)

(21) Appl. No.: 12/014,919

(22) Filed: Jan. 16, 2008

Related U.S. Application Data
(60) Provisional application No. 60/881,245, filed on Jan. 19, 2007.

(51) Int. Cl. A61L 9/00

(52) U.S. Cl. 422/120

(57) ABSTRACT

An arrangement for recharging device for a rechargeable device including a housing having an air inlet and an air outlet, an air movement device disposed within the housing and adapted to move air from the air inlet, through the housing, and out of the air outlet, an air sanitizing device disposed within the housing and capable of sanitizing air that flows through the housing, and a recharging interface adapted to electronically couple the rechargeable device with a power source to recharge a battery of the rechargeable device.

### RELATED APPLICATIONS

This application claims priority to, and the benefit of, U.S. Provisional Patent Application Ser. No. 60/881,245, filed Jan. 19, 2007 for AIR STERILIZING AND RECHARGING BASE AND RECHARGEABLE DEVICE ARRANGEMENT, the entire disclosure of which is fully incorporated herein by reference.

### BACKGROUND

Air contamination from airborne chemical and/or biological contaminants can cause a wide variety of human health complications. Chemical contaminants may include, but are not limited to, formaldehyde, aerosols, toluene, hydrocarbons, and carbon monoxide. These chemical contaminants can cause such health complications as eye irritation, headaches, nose and/or mucosal irritation, and fatigue. Biological contaminants may include, but are not limited to, bacteria, fungi, fungi spores, protozoa, viruses, algae, pollen, and various antigenic agents. Biological contaminants can cause such health complications as pneumonia, fever, mycotoxicosis, various infections, asthma, and other lung or breathing related alignments.

Rechargeable battery powered hand held devices are prevalent. These devices may include, but are not limited to, vacuum cleaners, electric toothbrushes, razors, shop tools (drills, saws, etc.), and portable entertainment/communication devices (cell phones, digital assistants, music players, etc.). Often, a charging/recharging base is sold with, or is mated with, such hand held devices, to charge/recharge the battery of the devices. The base may plug into an electrical main wall receptacle to provide the electric charge.

### SUMMARY

The arrangement disclosed in the present application is configured to combine a charging/recharging function for a rechargeable device with an air sanitizing function to provide a more healthy and less infection-prone environment. The arrangement may include a housing having an air inlet and an air outlet, an air movement device disposed within the housing and adapted to move air from the air inlet, through the housing, and out of the air outlet, an air sanitizing device disposed within the housing and capable of sterilizing air that flows through the housing, and a recharging interface adapted to electronically couple the rechargeable device with a power source to charge/recharge a battery of the rechargeable device.

In one embodiment the arrangement may include a ultraviolet (UV) radiation source, such as a UV-C light bulb, for example, disposed within an inner chamber on the housing. The fan disposed in the housing is positioned to move the air through the chamber such that the air is exposed to the UV radiation and then exhaust the air out of the air outlet. In another embodiment, a single source of power, such an electrical outlet, is used to power both the charging/recharging function for a rechargeable device and the air sanitizing function.

Further aspects and concepts will become apparent to those skilled in the art after considering the following description and appended claims in conjunction with the accompanying drawings. The embodiments described in this summary and throughout the specification are not intended to limit the meaning or scope of the claims in any way. The terms used in the claims have all of their full ordinary meaning.

### BRIEF DESCRIPTION OF THE DRAWINGS

In the accompanying drawings, which are incorporated in and constitute a part of the specification, embodiments of the invention are illustrated, which, together with a general description of the invention given above, and the detailed description given below, serve to exemplify example embodiments of the invention.

FIG. 1 is schematic representation of an exemplary embodiment of an air sanitizing and charging/recharging base and a rechargeable device arrangement as disclosed in the present application;

FIG. 2 is a front view of a second exemplary embodiment of an air sanitizing and charging/recharging base and a rechargeable device arrangement as disclosed in the present application;

FIG. 3 is a side view of the exemplary arrangement of FIG. 2;

FIG. 4 is a side view of the exemplary arrangement of FIG. 2 showing components within a base housing;

FIG. 5 is a perspective view of components of an air sanitizing portion of the exemplary arrangement of FIG. 2;

FIG. 6 is a perspective view of a base of the exemplary arrangement of FIG. 2; and

FIG. 7 is a perspective view of a rechargeable device of the exemplary arrangement of FIG. 2.

### DETAILED DESCRIPTION

The present application discloses an air sanitization charging/recharging base and rechargeable device arrangement. While the exemplary embodiments illustrated and described herein are presented in the context of a rechargeable hand held vacuum and a charging/recharging base that includes an air sanitizing portion having a fan and an ultraviolet-C (UVC) bulb, those skilled in the art will readily appreciate that the present invention may be used and configured in other ways. For example, the air sanitization portion may include any suitable means, alone or in combination, for reducing airborne contaminants, such as for example, ultraviolet irradiation, nanosilver coatings, ozone generation, anti-microbial filtration, air ionization, or the like. In addition, any suitable method of moving air through the arrangement may be used, such as but not limited to a fan, a blower, an air pump, or other suitable device. Furthermore, the arrangement may be configured to recharge any suitable rechargeable device, such as for example, but not limited to, rechargeable tools (e.g. drills, grinders, etc.), rechargeable communications devices (e.g. cell phones, nursery monitors, digital assistants, music players, etc.), rechargeable personal care devices (e.g. tooth brushes, razors, etc.), rechargeable homecare devices (e.g. hand held vacuum) and battery packs.

While various aspects and concepts of the invention are described and illustrated herein as embodied in combination in the exemplary embodiments, these various aspects and concepts may be realized in many alternative embodiments, either individually or in various combinations and sub-combinations thereof. All such combinations and sub-combinations are intended to be within the scope of the present invention. Still further, while various alternative embodiments as to the various aspects and features of the invention, such as alternative materials, structures, configurations, methods, devices, circuitry, and so on may be described herein, such descriptions are not intended to be a complete or exhaustive list of available alternative embodiments, whether presently known or identified herein as conventional or standard or later developed. Those skilled in the art may readily adopt one or more of the aspects, concepts or features of the invention into additional embodiments within the scope of the present invention even if such embodiments are not expressly disclosed herein. Additionally, even though some features, concepts or aspects of the invention may be described herein as being a preferred arrangement or method, such description is not intended to suggest that such feature is required or necessary. Still further, exemplary or representative values and ranges may be included to assist in understanding the present invention however, such values and ranges are not to be construed in a limiting sense and are not intended to be critical values or ranges. The embodiments described in the summary and throughout the specification are not intended to limit the meaning or scope of the claims in any way. The terms used in the claims have all of their full ordinary meaning.

As used in this application, air sanitizing refers to removing all or some of the contaminants from the air (i.e. generally reducing or eliminating the presence of contaminants) and/or inactivating or rendering useless the contaminants (e.g. prohibiting growth and reproduction of the contaminants). As used in this application, charging may refer to providing an initial charge or to recharging a battery or other electric storage device. The charging function may be adapted to charge and/or recharge a specific rechargeable device or may be adapted to provide adaptability to charge and/or recharge a variety of rechargeable devices.

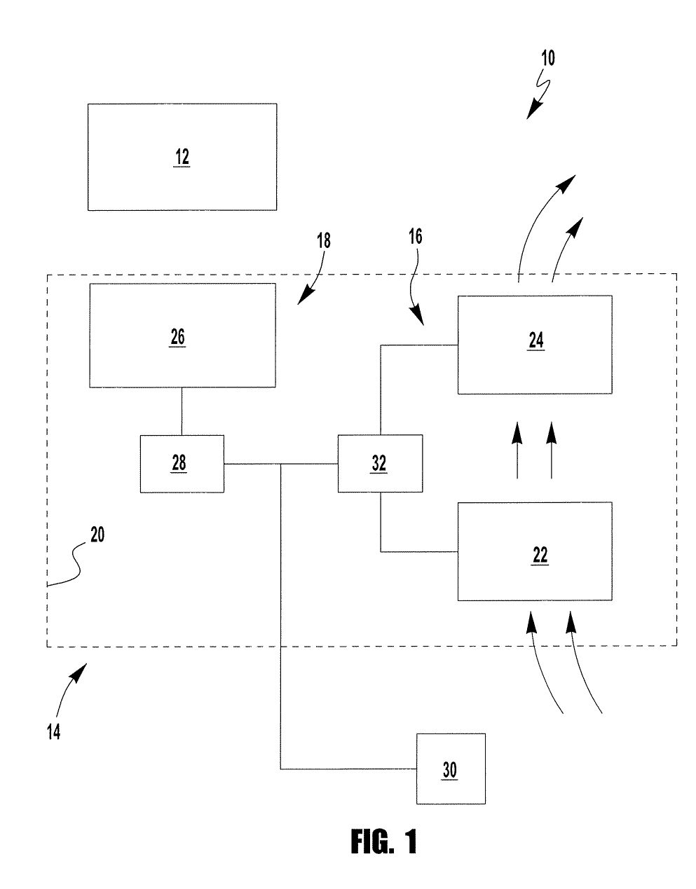

FIG. 1 is a schematic representation of an exemplary embodiment of an air sanitization charging/recharging base and rechargeable device arrangement 10. The arrangement 10 includes a rechargeable device 12 and a base 14 having an air sanitizing portion 16 and a charging/recharging portion 18 located within a housing 20. The air sanitizing portion 16 includes an air movement device 22 and an air sanitizing device 24. The air movement device 22 is adapted to move air through the housing 20, and past and/or through the air sanitizing device 24. A variety of air movement devices 22 may be used depending on the application, the type of air sanitizing device 24 and method, and other factors. In one embodiment, the air movement device 22 includes a fan, disposed within the housing 20, that is capable of drawing air into the housing 20 and moving the air past and/or through the air sanitizing device 24. In another embodiment, the air movement device 22 includes a connection to an external source of pressurized air and suitable airflow passages to allow the air to flow through the housing 20.

The air sanitizing device 24 may be any suitable device capable of removing, killing, and/or reducing the amount of contaminants in the air that passes through the arrangement 10. As non-limiting examples, the air sanitizing device 24 may include, alone or in combination, a germicidal lamp, an ozone generator, a nanosilver coated surface or filter, a HEPA filter (high efficiency particulate air filter), an activated carbon source or the like, or other suitable means for sanitizing and/or removing contaminants from the air.

The charging/recharging portion 18 may include a charging interface 26 for electrically communicating with a rechargeable device 12 and a charging circuit 28. The charging/recharging interface 26 may be configured in any suitable manner to provide a charge to a select rechargeable device or devices. For example, it is known to have metal contacts on the rechargeable device 12 that engage metal contacts on the charging/recharging interface 26 when the device is placed on the base 14. The engagement between the contacts on the device 12 and the contact on the interface 26 create an electrical path or connection between the device and the base 14 that allows the base to supply the device with electrical current to recharge the device's batteries. As a further example, it is also known to use inductive charging, where instead of metal contacts, the base 14 and device 12 form a two-piece transformer when the device is placed in the base in order to recharge the device. The device recharging portion 18 of the arrangement 10 is not limited to one specific method of charging/recharging, but may instead, use any suitable known or newly developed method(s) for charging/recharging a rechargeable device.

The arrangement 10 includes a connection to a power supply 30 or may include an internal power supply. For example, recharging units are known to utilize an AC to DC adapter that is plugged into an electricity main receptacle (120 VAC) in a home to supply DC power to the charging circuit 28 and recharging interface 26. Any suitable power supply, however, may be used. The power supply 30 may also be contained within the housing 20 or external to the housing. The air sanitizing portion 16 may include a circuit board 32 for supplying power to the air sanitizing device 24 and/or the air movement device 22, as needed. The power supply 30 for the recharging portion 18 may also be used to power the air sanitizing portion 16 or the air sanitizing portion may have a separate power supply. Thus, for example, if an AC to DC adapter is used for the recharging portion 18, the air sanitizing circuit 32 and components may be adapted to operate on the DC power from the adapter. In this manner, a single power supply may be used to provide both the recharging function and air sanitizing function, thus saving space and electricity and reducing the number of components and appliances.

The device 12 may be any suitable rechargeable device compatible with the recharging portion 18 of the arrangement 10. The device 12, for example, may be, but not limited to, rechargeable tools, rechargeable communications devices, rechargeable personal care devices, rechargeable homecare devices and battery packs.

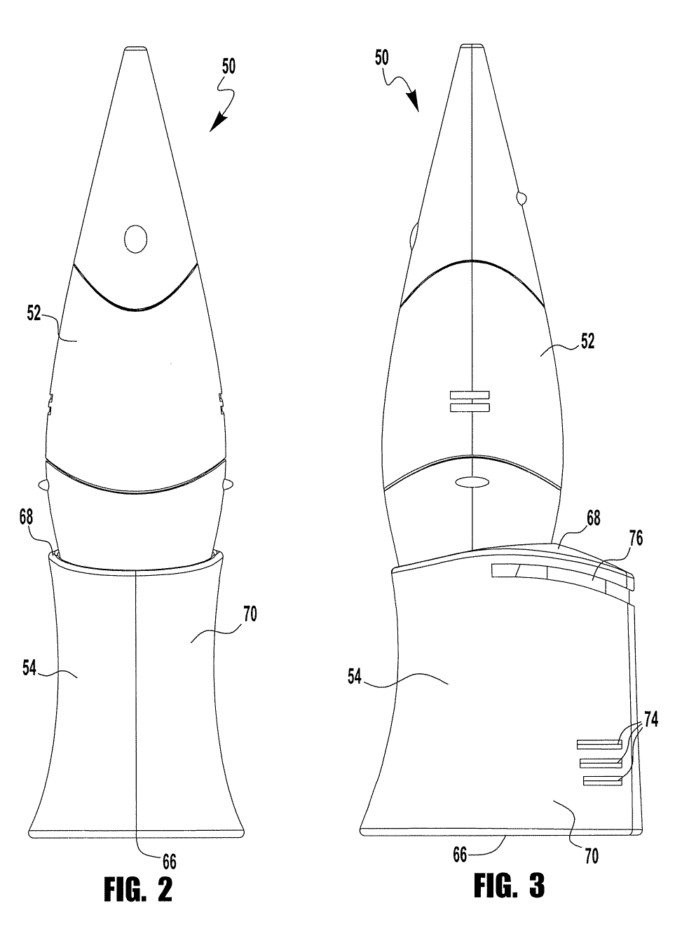

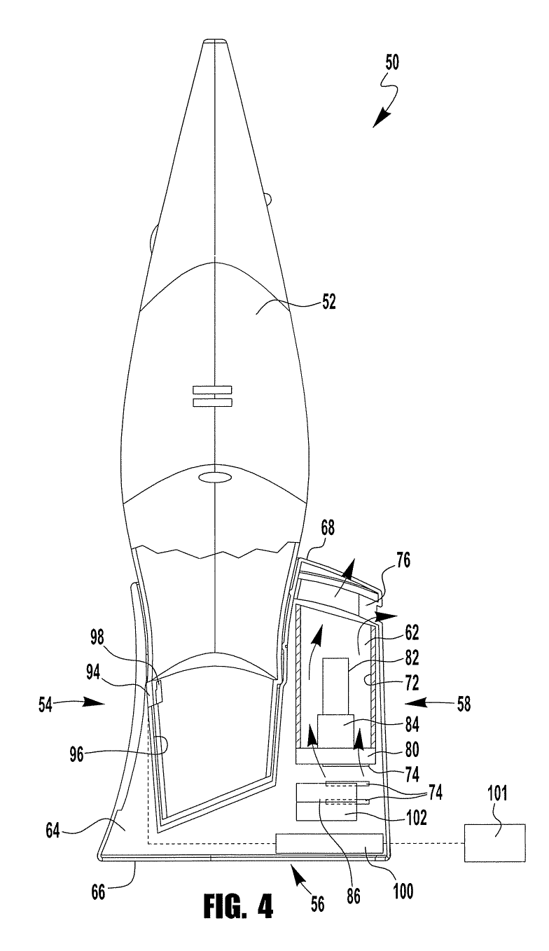

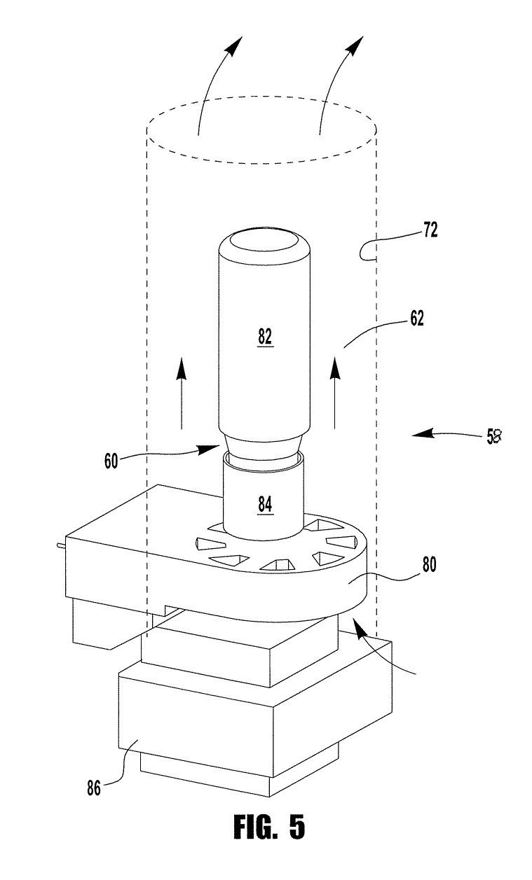

FIGS. 2-7 illustrate a second exemplary embodiment of an air sanitization charging/recharging base and rechargeable device arrangement. The arrangement 50 includes a rechargeable device 52 and a base portion 54. Referring to FIG. 4, the base portion 54 includes a recharging portion 56 and an air sanitizing portion 58 that is configured and adapted for UV germicidal irradiation. The air sanitizing portion 58, in the depicted embodiment, includes a UV radiation source 60 (FIG. 5) associated with an inner chamber 62 of the base portion 54 for providing ultraviolet germicidal irradiation of contaminants in the air that passes through the chamber.

The base portion 54 includes a housing 64 having a substantially flat bottom surface 66, a top surface 68, and a generally curved, continuous side surface 70 forming a generally ovate cross sectional shape. The housing 64, however, may be configured as desired and is not limited to the shape or configuration of the exemplary embodiment of FIGS. 2-7. For example, the substantially flat bottom surface 66 in the depicted embodiment may allow the arrangement 50 to stably sit on a flat surface, such as for example a floor or table top. The arrangement 50, however, may be configured without the flat bottom surface and may instead be adapted to mount to a vertical or horizontal surface or directly plug-in to electrical receptacle on a wall, for example. As a further example, the cross-sectional shape of the housing 64 may be any suitable shape, such as for example, but not limited to, circular, polygonal, oval, or the like.

The housing 64 has one or more inner walls or surfaces 72 (see FIG. 4) that form one or more inner cavities in which the components of the charging portion 56 and the air sanitizing portion 58 are disposed. Regarding the air sanitizing portion 58, the housing 64 includes one or more air inlet vents 74, through which a contaminated stream of air may enter the base 54. The inner wall 72 of the housing 64 forms the inner chamber 62 associated with the UV radiation device 60. The inner chamber 62 is in fluid communication with the air inlet vents 74 and one or more air exhaust vents 76, such that air that enters the base 54 via the air inlet vents 74 flows through the inner chamber 62 and out of the air exhaust vents 76.

The air inlet vents 74 may be provided in any suitable configuration. In the depicted embodiment, the air inlet vents 74 are configured as two groups of three horizontal slots, each group located toward the air sanitization portion 58 on the side surface 70 near the housing's bottom surface 66. The air exhaust vents 76 may be provided in any suitable configuration. In the depicted embodiment, the air exhaust vents are configured as a generally horizontal groove on the side surface 70 near the housing's top surface 68. The air exhaust vents 76 may be configured to suitably direct the outgoing air stream away from the base portion 54. Thus, air flows through the base 54 generally from bottom to top.

The arrangement 50 includes a device for moving air through the chamber 62, such as but not limited to a fan or air pump. In the depicted embodiment, the device is realized as a DC-powered fan 80 adapted to draw air into the air inlet vents 74. A fan utilizing DC-power provides a convenient means of moving the air through the base 54 since the recharging portion 56 may operate on DC power and utilize an AC to DC adapter. A 1.08 W fan powered by a brushless DC motor and including a thermoplastic impeller and housing, with an airflow of approximately 6 to 9 CFM in free air has been found suitable. Any suitable device capable of providing sufficient air movement through the housing 64, however, may be used.

The fan 80, may be positioned in any suitable location that, when active, allows the fan to cause air flow through the base 54. In the depicted embodiment, the fan 80 is disposed at least partially within the housing 64, relatively above the air inlet vents 74, and relatively at the bottom of the inner chamber 62. When operating, the fan 80 draws air through the air inlet vents 74, forces the air through the inner chamber 62 and subsequently out the air exhaust vents 76. In other embodiments, the fan 80 may be above the base or external to the housing.

The UV radiation source 60 associated with the inner chamber 62 is adapted to irradiate the air passing through the inner chamber to remove, kill, and or reduce the contaminants in the air. The UV radiation source 60 includes a bulb 82 and a socket 84 disposed, at least partially, within the inner chamber 62. The bulb 82 may be a known, an adapted, or a newly developed bulb capable of emitting ultraviolet radiation, although any suitable bulb may be employed, such as for example a cold cathode fluorescent bulb. The bulb 82 may be configured to emit any suitable type of UV radiation, such as UV-A, UV-B, UV-C, or may be configured to simultaneously emit two or more types of UV radiation. In the depicted embodiment, the bulb 82 is configured to emit UV-C radiation in a wavelength, for example, of approximately 200-280 nanometers (nm) and preferably in a wavelength of approximately 254 and/or 253.7 nm. A 3-watt, UV-C bulb, model no. GTL-3, available from USHIO America, Inc. has been found suitable. A bulb, however, emitting any suitable wavelength may be employed. Furthermore, more than one bulb may be used within the base 54.

The bulb 82 may include other aspects that improve its performance characteristics. For example, the bulb 82 may include a high purity synthetic quartz glass envelope. It may also include a clear coating on the inside for decreasing depreciation of the UV-C radiation output. Furthermore, the bulb 82 may be configured to produce ozone.

The socket 84 is used to maintain the desired orientation of the bulb 82 within the housing 64 as well as electronically coupling the bulb to a power supply. The socket 84 may be a known, an adapted, or a newly developed socket suitable for the application. The socket 84 and the fan 80 may be coupled to a ballast 86 or similar feature to regulate the current flow to the bulb 82 and fan.

The inner chamber 62 may be configured in any suitable manner. Any configuration that allows air passing through the chamber to be exposed to UV radiation to remove, kill, and or reduce the contaminants in the air may be used. In the depicted embodiment, inner chamber 62 has a generally circular cross section; thus, the inner wall 72 of the inner chamber is generally a continuous curved surface, without sharp corners, which may be beneficial for reducing air flow obstructions within the chamber. The inner chamber 62, however, may be configured in a variety of ways. For example, though preferably a curved surface, such as for example an elliptical, an oval, or a circular cross section, any suitable configuration may be used, such as for example a rectangular or polygonal cross section.

The bulb 82 and socket 84 are disposed at least partially within the inner chamber 62. For example, in the depicted embodiment, the bulb 82 and socket 84 are generally positioned within the inner chamber 62 along the chamber's midpoint, thereby enabling the airstream that flows through the inner chamber to generally envelop the bulb as it flows past.

The inner chamber 62 may include various materials disposed along the inner wall 72 for increasing the effectiveness of the UV radiation source 60. In one embodiment, the inner chamber 62 includes a reflective surface, such as for example aluminum, titanium, or the like, for increasing the intensity and/or density of distribution of the radiation within the inner chamber 62. In the depicted embodiment, the inner wall 72 is made, in whole or in part, of aluminum and may include an inner layer of titanium oxide (TiO₂). TiO₂ acts as a photocataylst capable of decomposing organic compounds when activated by UV radiation. In particular, UV radiation causes the formation of hydroxyl (—OH) free radicals that are efficient oxidizers of organic matter, such as microorganisms. The layer of TiO₂ is disposed along the inner wall surface 72 such that it substantially encircles the UV radiation source 60 to maximize the UV radiation contact with the TiO₂ layer. In addition, TiO₂ may be suitably applied to other components or air sanitizing devices that may be used by the arrangement 50 and exposed to UV radiation, such as for example, filter elements or screens.

Short wave UV light may be harmful to humans in that it can produce inflammation of the cornea of the eye, which can lead to vision impairment. The air inlet vents 74 and the air exhaust vents 76 are positioned and configured such that the UV light is not visible from outside of the base 54. In addition, the base 54 may optionally include safety features that block and/or prevent direct line of sight to the UV radiation source 60. For example, baffles (not shown) may be used around the air exhaust ports 76, for example, to provide a visual barrier between the ports and the UV radiation source 60. Use of such baffles may also provide a performance benefits in some applications. In particular, the baffles may act as a retardant that suitably increase the duration of time the air is within the inner chamber 62 being exposed to the UV radiation, thereby increasing the extent of germicidal activity. Use of baffles, however, is not required. Sufficient exposure time of the air may be achieved via the fan 80 selection and inner chamber 62 configuration.

Referring to the recharging portion 56 of the base 54, the housing 64 forms a engagement or docking area 90 for receiving the rechargeable device 52. In the depicted embodiment, the housing 64 forms an opening 92 for receiving the rechargeable device 52. The opening 92 forms a generally cylindrical depression configured to generally match the external shape of a portion of the rechargeable device 52. The docking engagement area 90, however, may be configured in any suitable manner to allow recharging of the rechargeable device 52, and preferably, retaining the device on or in the base 54.

The base 54 includes a charging interface 94 for electronically coupling the base to the rechargeable device 52. In the depicted embodiment, the charging interface 94 is realized as one or more metal contacts disposed on the surface 96 of the depression. The rechargeable device 52 includes a corresponding charging interface 98. In the depicted embodiment, the charging interface 98 is realized as corresponding metal contacts that engage the charging interface 94 on the base 54 when the rechargeable device 52 is inserted into the opening 92.

The charging interface 94 is in circuit communication with a charging circuit board 100 disposed within the housing 64. The arrangement 50 is adapted to receive power from a power supply 101. The charging circuit board 100 is designed to route DC power from the power supply 101 to the charging interface 94. The power supply 101 may be internal to the arrangement 50, such as a battery, for example, or may be external to the arrangement, such as an electrical outlet. The arrangement 50 may utilize an AC to DC adapter (not shown), as is known in the art. The adapter may be placed in circuit communication with the base portion 54 by, for example, an electrical wire, and may include a plug for connecting to an electrical outlet. Any AC to DC adapter may be used that is suitable for use with the specific charging circuit 100 and rechargeable device 52. For example, in one embodiment, an adapter capable of receiving 120 VAC and outputting about 9 VDC is suitable.

The air sanitizing portion 58 includes a circuit board 102 for supplying power to the UV radiation source 60 and/or the fan 80, as needed. The power supply 101 for the recharging portion 56 may also be used to power the air sanitizing portion 58 or the air sanitizing portion may have a separate power supply. Thus, for example, if an AC to DC adapter is used for the recharging portion 56, the air sanitizing circuit 102 and components may be adapted to operate on the DC power from the adapter or convert the DC power back to AC power. In this manner, a single power supply 101 may be used to provide both the recharging function and the air sanitizing function, thus saving space and electricity and reducing the number of components and appliances.

The rechargeable device 52, in the depicted embodiment, is realized as a battery powered, hand held vacuum cleaner. The rechargeable device 52, however, may be any rechargeable device suitable for charging via the recharging portion 56 and recharging circuitry 100.

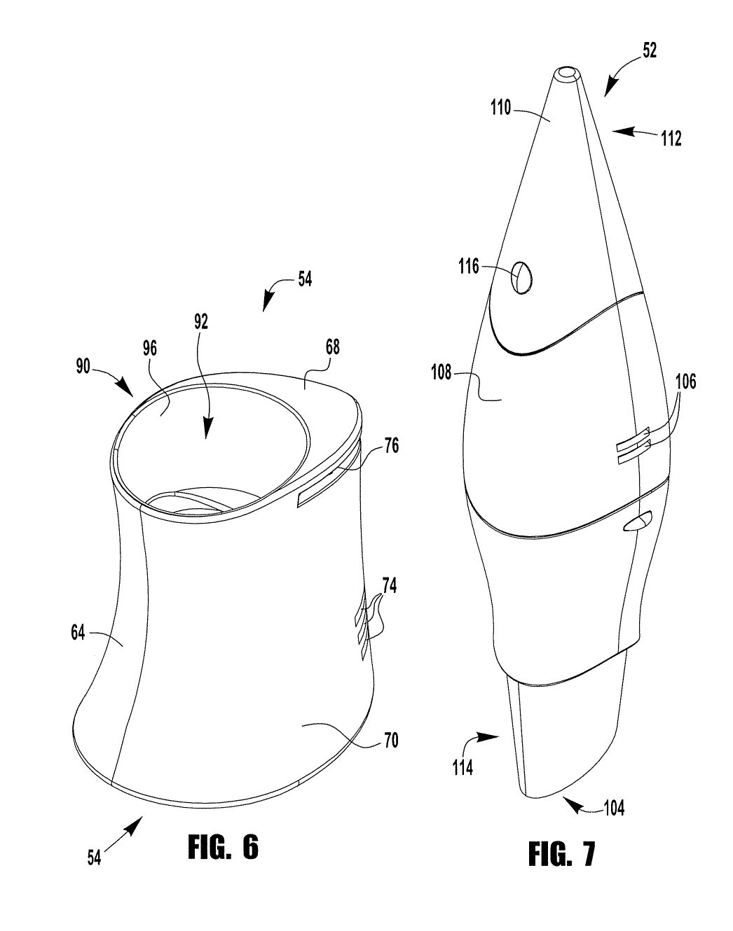

Referring to FIG. 7, the vacuum cleaner 52 may be configured to include operating components as are known in the art. For example, the hand held vacuum 52 may include an intake port 104, one or more exhaust ports 106, a fan (not shown) for creating suction, an electric motor (not shown) for powering the fan, a rechargeable battery pack (not shown) for supplying power to the motor, a porous bag (not shown) for filtering and collecting contaminants from the air, and a housing 108 that contains all the components. The housing 108 may includes a handle portion 110 adapted to be gripped by hand. In the depicted embodiment, the handle portion 110 is located at a first end 112 of the vacuum cleaner 52 and the intake 104 is located at a second end 114. The housing 108 may also include various control elements, such as an ON/OFF switch 116 for turning the vacuum on ON/OFF. The housing 108 may comprise removable sections to allow access to the internal components for replacing the bag, or replacing or fixing other components.

To charge the vacuum cleaner 52, the second end 114 of the vacuum is inserted into the opening 92 on the base 54. The opening 92 is configured to hold the vacuum 52 securely in place and engage the contacts 98 on the vacuum with the contacts 94 on the inner surface 96 of the depression. Once engaged, the contacts 94 on the base 54 allow power to be transferred to the device.

The invention has been described with reference to the preferred and specific embodiments. Modification and alterations of these embodiments will occur to others upon a reading and understanding of this specification. It is intended to include all such modifications and alterations within the scope of the disclosure described herein. The embodiments described in the summary and throughout the specification are not intended to limit the meaning or scope of the claims in any way. The terms used in the claims have all of their full ordinary meaning.

1. A charging device, comprising
a housing having an air inlet and an air outlet;
an air movement device disposed within the housing, the air movement device adapted to move air from the air inlet, through the housing, and out of the air outlet;
an air sanitizing device disposed within the housing and capable of sanitizing air that flows through the housing; and
a charging interface adapted to couple a rechargeable device with a power source to charge/recharge a battery of the rechargeable device.

2. The charging device of claim 1 further comprising an internal chamber in fluid communication with the air inlet and the air outlet, wherein the air sanitizing device is disposed within the internal chamber.

3. The charging device of claim 1 wherein the air sanitizing device comprises an ultraviolet radiation source.

4. The charging device of claim 3 wherein the ultraviolet radiation source includes a ultraviolet-C light bulb.

5. The charging device of claim 3 wherein the internal chamber includes a generally cylindrical, reflective side surface.

6. The charging device of claim 1 wherein the air movement device comprises a fan disposed between the air inlet and the air outlet.

7. The charging device of claim 1 further comprising a docking area having an opening adapted to receive a portion of the rechargeable device.

8. The charging device of claim 7 wherein the docking area comprises a depression in the housing adapted to generally conform to the outer contour of the rechargeable device

9. The charging device of claim 1 wherein the device is adapted to use a single power source to provide power to the air sanitizing device and the rechargeable device.

10. A charging device and a rechargeable device arrangement, comprising:
a rechargeable device having a battery and a first charging interface;
a base comprising:
a housing having an air inlet and an air outlet;
an air movement device disposed within the housing, the air movement device adapted to move air from the air inlet, through the housing, and out of the air outlet;
an air sanitizing device disposed within the housing that sanitizes air within the housing; and
a second charging interface adapted to form an electrical connection with the first charging interface; and
a power source electrically coupled to the second charging interface to charge/recharge the battery of the rechargeable device when the first charging interface and the second charging interface form an electrical connection.

11. The arrangement of claim 10 further comprising an internal chamber in fluid communication with the air inlet and the air outlet, wherein the air sanitizing device is disposed within the internal chamber.

12. The arrangement of claim 10 wherein the air sanitizing device comprises an ultraviolet-C light bulb.

13. The arrangement of claim 10 wherein the air movement device comprises a fan disposed between the air inlet and the air outlet.

14. The arrangement of claim 10 further comprising a docking area having an opening adapted to receive a portion of the rechargeable device.

15. The arrangement of claim 14 wherein the second recharging interface is disposed along a surface of the docking area.

16. The arrangement of claim 10 wherein the rechargeable device is a hand-held vacuum cleaner.

17. An arrangement for charging a rechargeable device, comprising
a housing defining an internal chamber, the housing comprising:
an air inlet in fluid communication with the internal chamber;
an air outlet in fluid communication with the internal chamber; and
a docking portion adapted to engage the rechargeable device;
an ultraviolet bulb disposed within the internal chamber;
a fan disposed within the housing, the fan adapted to draw air into the housing through the air inlet and move the air past the ultraviolet bulb and out of the air outlet; and
a charging interface disposed along a surface of the docking portion, the charging interface adapted to couple the rechargeable device with a power source to charge/recharge a battery of the rechargeable device.

18. An arrangement for charging a rechargeable device, comprising
a housing;
a means for flowing air through the housing;
a means for sanitizing the air that flows through the housing; and
a means for charging a battery of the rechargeable device.

19. The arrangement of claim 18 wherein the means for sanitizing the air further comprises a means for creating ultraviolet radiation.

20. A vacuum cleaner with a charging base, comprising:
a base having an air inlet and an air outlet;
an air movement device disposed within the base, the air movement device adapted to move air from the air inlet, through the base, and out of the air outlet;
an air sanitizing device disposed within the base and capable of sanitizing air that flows through the base; and
a charging interface adapted to couple a rechargeable device with a power source to charge/recharge a battery of the rechargeable device.
